(19) United States
(12) Patent Application Publication
(10) Pub. No.: US 2002/0068053 A1
(43) Pub. Date: Jun. 6, 2002

(54) Process for the production of anti-tumor Th1 cells

(76) Inventors: Martin Rocken, Muenchen (DE); Oliver Egeter, Muenchen (DE); Ralph Mocikat, Muenchen (DE)

Correspondence Address: Helfgott & Karas, P.C., Suite 6024, 350 Fifth Avenue, New York, NY, 10118, US

(21) Appl. No.: 09/932,575

(22) Filed: Aug. 16, 2001

Related U.S. Application Data
(63) Continuation of application No. PCT/EP00/01339, filed on Feb. 17, 2000.

(51) Int. Cl. A61K 45/00; C12N 5/08; C12N 15/01; A61K 31/407

(52) U.S. Cl. 424/93.71; 435/372; 435/443; 435/446; 514/410

(57) ABSTRACT

The invention relates to a pharmaceutical preparation comprising tumor-reactive cells similar to Th1 which produce large quantities of interferon γ and produce little or no interleukin 4; together with pharmaceutically compatible excipients and auxiliary agents. The invention also relates to a method for producing tumor-reactive cells similar to Th1 which produce large quantities of interferon γ and produce little or no interleukin 4.

The invention relates to a process for the production of anti-tumor Th1 cells that produce interferon-γ (IFN-γ).

The immune system of mammals, including that of humans, has a number of strategies to protect it against various tumors or even to combat them. For example, it is possible to provoke an immune response to tumor antigens, i.e. (glyco) proteins that are expressed specifically at the surface or inside tumor cells. An immune response to these antigens can provide protection before solid tumors and tumors of the hematopoietic system are established. From the literature, it is known that most immune responses to tumors are dependent both on the CD4+T helper cells (Th) and on the cytotoxic CD8+T cells (Tc). However, it has been recognized that the cytotoxic Tc are of particular significance for an effective tumor defense. The role of the Th was not extensively studied in this regard until now.

Numerous medications and processes for immunization against various tumor types have been developed. For example, immunization with the dendritic antigen-presenting cells (APC), co-application of pro-inflammatory mediators, particularly interleukin-(IL)-12 and T cell activation by means of modulation of the CTLA4 molecule have been proven to be particularly effective. Additional strategies include vaccination with preparations that contain either tumor-infiltrating T lymphocytes (TIL), lymphokin-activated killer cells (LAK), or CD8+ T lymphocytes. However, such approaches have proven to be effective only within certain limitations.

Although numerous immunization protocols for tumor prevention have been described, there have been hardly any medications until now that act via the immune system against tumors that have already been established. The only really effective therapy protocol is the adoptive transfer of allogenic T lymphocytes in the treatment of various forms of leukemia. The data from the past twenty years have documented that T lymphocytes that produce IFN-γ play an important role in cellular tumor defense. Until now, particularly those tumor immune responses that are directed specifically against antigens that are presented at Tc via the main histocompatibility antigens of Class I (MHC I) have been studied. MHC Class II-restrictive Th are viewed as being regulator cells. Their presence appears to be important for efficient tumor prevention. Nevertheless, neither their precise role in the anti-tumor immune response nor their target peptides, aside from a few exceptions, are known (Immunity 95: 2, 45-57). Because of these difficulties, they have not been developed as medications in the sense of immune therapies against specific tumors.

WO99/51720 describes a very special process (subsequently published), with which Th1 cells can be generated. However, it does not allow the development of effective CD2-positive cells or T cells that specifically recognize an antigen, such as a tumor antigen.

A known process (Kuge et al., 1995, J. Immunol. 154(4), 1777-85) describes that Th1 lymphocytes can lyse tumor cells in a culture dish. However, there are no indications in this reference of any in vivo therapy. Also, this approach cannot be directly transferred to the in vivo situation.

The present invention is based on the task of making available a process for the production of a preparation for the prevention and treatment of tumor diseases. In particular, what is involved is developing specifically tumor-reactive cells in vitro, which will be effective against tumors in vivo. The goal of every tumor therapy is to destroy the tumor as much as possible, and at the same time to spare all the other cells of the body. Traditional therapies do not sufficiently achieve this goal. For this reason, attempts have been made to develop specific tumor therapies. The only therapy approach that is tumor-specific according to our present understanding is an immune therapy, since the lymphocytes only recognize those target cells against which they have been activated, due to their antigen receptor.

Because of their ability to directly kill tumor cells, current research is predominantly betting on an anti-tumor therapy using tumor-specific Tc. However, it is known from autoimmune diseases that it is not Tc but rather Th that trigger autoimmune diseases in particularly dramatic manner (Racke et al., 1994). Particularly serious forms of autoimmune diseases, which often have an acute fatal outcome, are triggered by the adoptive transfer of Th that produce IFN-γ, which are also called Th1. Since these adoptively transferred Th1 destroy the target organ but completely spare all the other tissue, the adoptive transfer of tumor-specific Th1 should be particularly well suited for tumor therapy.

The preparation produced according to the invention is supposed to provide protection against possible tumor development, and, in particular, is also supposed to be effective against tumors that have already been established. Furthermore, the preparation produced according to the invention is supposed to be able to be adapted to the tumor to be combated in each instance. The preparation is supposed to imitate or reinforce the natural mechanisms of tumor defense, and therefore possess no side effects, or only few side effects.

This task is accomplished by a process for the production of a pharmaceutical preparation that contains tumor-specific Th, which are characterized by a cytokin pattern similar to Th1, in other words characterized by a high IFN-γ production P1 (100≦P1≦100,000 U/ml) and a low IL-4 production P2 (P2≦1000 U/ml). In this connection, the P1:P2 ratio is of particular significance, and should be on the order of 10:1 or higher. The T lymphocytes are administered together with pharmaceutically compatible excipients and processing aids. A process for the production of tumor-specific Th1 cells is established. These are cells that recognize the tumor or tumor-associated antigens (amino acids/protein sequences) and react to them. However, they demonstrate no or hardly any reactivity with regard to other antigens that are efficiently presented in the body. The invention also relates to the use of these Th1 for the prevention and/or treatment of solid tumors and tumors of the hematopoietic system.

Analogous to the experiments concerning the pathogenesis of autoimmune diseases, it was now found that Th, particularly Th1 and cells similar to Th1, possess a significant importance in tumor defense. By culturing Th1 that specifically recognize (glyco) proteins of the tumor to be combated, effective medications for the prevention/treatment of the tumor can be produced. Preparations that contain these cells are administered to the organism by the parenteral path, mostly by intraperitoneal, intravenous, or subcutaneous administration. The Th1 that are administered activate or reinforce the immune response that is directed against the tumor, either by direct induction of cell death, or by production of their specific cytokins. These can either be directly cytotoxic for the tumor, or can have the effect that effector cells that are toxic for the tumor, Tc or macrophages, are recruited and activated. As a result, they have a decisive share in the immune-mediated remission or regression of the tumor.

According to the invention, Th1 cells against any desired types of tumors can be produced. The production of Th1 effective against lymphomas is preferred, but solid tumors are also important target structures. The corresponding tumor types can therefore be combated with the preparation produced according to the invention. The method and posology of administration are determined by a physician, in accordance with the type and severity of the tumor to be treated, the condition of the patient, and the period of therapy. The preparation produced according to the invention is administered in a dosage of 1×10⁶-100-1000×10⁶ cells/kg of body weight, in a one-time dose or repeatedly. The preparation can be administered both to humans and to animals.

The Th1 cells produced according to the invention can protect against hematopoietic tumors; they are also suitable for the treatment of established lymphoma, and even solid tumors can at least be controlled with such Th1 cells.

The process according to the invention furthermore allows obtaining tumor-specific Th1 cells also from animals that are already ill with an advanced tumor. In this connection, it is possible to cause precisely those few families of T lymphocytes that either specifically or primarily recognize the tumor to grow, from among the large number of different immune cells. By means of adoptive transfer of tumor-specific Th1 cells, a very quickly effective anti-tumor immune response can be established, which is then also very quickly effective in therapy and, at the same time, is safe, i.e. free of side effects, to a great extent.

In order to investigate the role of tumor-reactive MHC Class II-restrictive CD4+Th in greater detail, it was necessary to develop a culture system that allows the generation of tumor-specific Th1. MHC Class II-restrictive tumor peptides have hardly become known as yet. For this reason, a system was developed that allows the establishment of tumor-specific Th1 without knowing specific tumor peptides. Tumor cells that have been inhibited in their proliferation by radiation or chemotherapeutics such as Mitomycin C are cultivated in vitro together with autologous/syngeneic APC and Th. For reasons of practicability, studies until now have been conducted with a B-cell lymphoma that can present its own tumor antigens via MHC II. CD4+Th were obtained from mice that already carry the A20-B-cell lymphoma, or also from mice that were immunized against this lymphoma. These Th were then cultivated in vitro, using the method described below, in such a way that tumor-specific Th1 are formed, Th that produce large amounts of IFN-γ, but no or hardly any IL-4. Measured using a commercially available ELISA test, effective IFN-γ levels were between 10² and 10⁵ U/ml; measured using CT4S cells, IL-4 had to be below 1000 U/ml. Again, it was important that the ratio of IFN-γ to IL-4 was ≧10:1. These tumor-specific Th1 were then used for tumor therapy.

### Material Production of Tumor-specific Th1

Murine A20 cells (ATCC-TIB-208) were cultivated in RPMI1640, which contained 10% FCS and 50 mM β-mercaptoethanol, at 37° C., in a water-saturated atmosphere and 5% CO₂. Before being used for the in vitro culture with the Th, the tumor cells were irradiated at 65 Gray.

Th (>95% purity) were obtained by negative selection, using Biotex-T-cell acids (Tebu, Frankfurt). Other processes for obtaining Th using positive or negative concentration are equally well suited. These Th were cultivated in vitro in Dubeccos MEM (culture medium) to which 10% FCS, penicillin and streptomycin, 50 μM β-mercaptoethanol, and MEM amino acids had been added, at 37° C., in a water-saturated atmosphere and 7.5% CO₂. Other media also allow the establishment of Th lines, some of them without the use of serum.

APC were concentrated by means of the treatment of BALB/c spleen cells with anti-CD4 and anti-CD8 antibodies and complement (Behring-Werke, Marburg). Before use for the in vitro culture of Th, they were irradiated at 30 Gray.

Oligonucleotide containing phosphothioate-modified CpG-dinucleotide (CpG-ODN 1668) was purchased from MWG (Munich) (sequence: 5′-TCC ATG ACG TTC CTG ATG CT-3′). Anti-IL-4 antibodies come from the Clone 11B11, human rIL-3 was purchased commercially. Such antibodies have also been developed against human IL-4; furthermore, Th1 can also be produced using the soluble IL-4 receptor molecule (Breit et al., 1996, Eur. J. Immunol. 26, 1860-1865).

### EXAMPLE FOR THE PRODUCTION OF THE TUMOR-SPECIFIC Th1

Day 1

First Stimulation

0.2×10⁶ Th+irradiated tumor cells (0.2×10⁶ A20)+0.3×10⁶ APC+anti-IL-4 antibodies (e.g. 10 μg/ml 11B11) +/− immune-stimulating oligonucleotides (e.g. 0.2 μM CpG-ODN 1668)+IL-2 in a 96-hole round-bottom culture plate, 200 μl volume, culturing started.

Day 2

Replacement of medium, anti-IL-4 antibodies, oligonucleotide and IL-2.

Day 4

The cell suspension is transferred to a 24-hole culture plate, and fed with a medium that contains IL-2.

Day 5-10

Every 2 days (Day 7, Day 9), the medium is replaced and the cells are fed with a medium that contains IL-2. Depending on the cell density, the culture is distributed among additional culture dishes.

Day 11

Second Stimulation of the Cells

0.1×10⁶ Th+0.2×10⁶ irradiated A20+0.3×10⁶ APC+anti-IL-4 antibodies +/− immune-stimulating oligonucleotides in a 96-hole round-bottom culture plate, 200 μl volume, culturing started.

Day 12

Replacement of medium, anti-IL-4 antibodies, oligonucleotide and IL-2.

Day 14

The cell suspension is transferred to a 24-hole culture plate, and fed with a medium that contains IL-2.

Day 15-20

Every 2 days (Day 17, Day 19), the medium is replaced and the cells are fed with a medium that contains IL-2. Depending on the cell density, the culture is distributed among additional culture dishes.

Day 21

Third Stimulation

As needed, the Th can be restimulated every 10-14 days, for example a third time on Day 21 and more often, in accordance with the protocol for the second stimulation (Day 11-20), and expanded further. Alternatively, they are utilized for therapy by means of adoptive transfer.

When checked, these lines express surface antigens that characterize activated Th. The Th lines generated in this way are:

(1) specific for the tumor, here A20 lymphoma, and

(2) T-cell lines of the Th1 type (cells similar to Th1).

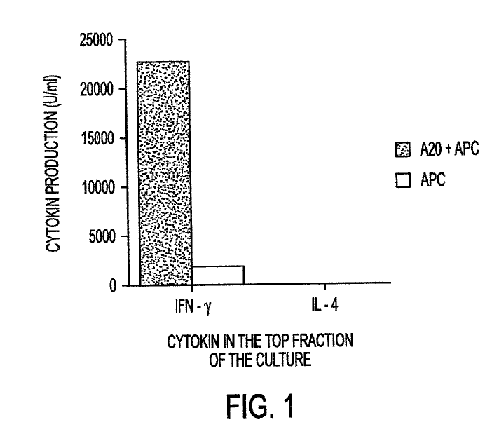

If they are stimulated in vitro with syngeneic spleen cells that present the tumor peptides of the A20 tumor via their MHC Class II molecules, they produce a lot of IFN-γ and little or no IL-4 (see production information on p. 6 and FIG. 1). However, if the Th are cultivated with syngeneic spleen cells that present no tumor antigens or antigens of a different tumor, that of MCP11-B-cell lymphoma, they produce no or only small amounts of cytokins. These cells represent pharmaceutical preparations that can be used in targeted manner for the treatment of A20 lymphoma.

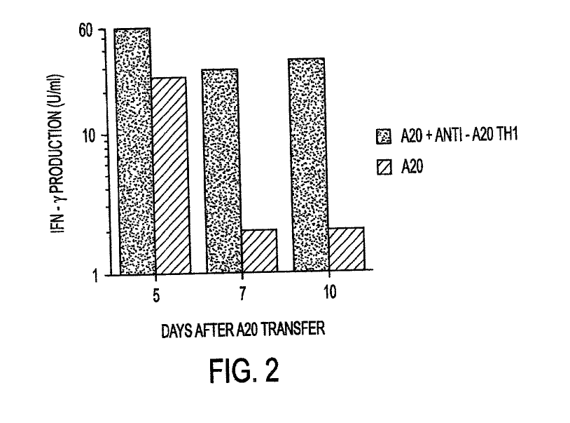

First, the effects of the adoptive transfer of A20-specific Th1 on the immune response of Balb/c mice to the A20 tumor was studied. For this purpose, 0.5×10⁶ A20 cells and 0.5×10⁶ A20-specific Th1 were injected into Balb/c mice intraperitoneally, at the same time. After 5, 7, and 10 days, the spleen cells were isolated from the mice, and stimulated in vitro with the tumor, here the 0.2×10⁶ A20 tumor, which was presented by syngeneic APC. T lymphocytes of mice to which tumor cells had been administered produced IFN-γ only if they were isolated within 5 days after tumor application. In contrast to this, T lymphocytes of mice that had been injected with both A20 tumors and A20-specific Th1 produced IFN-γ even if they were stimulated with syngeneic APC with tumor-specific antigens more than 10 days after the tumor transfer (FIG. 2). In this way, a prolonged Th1 immune response to the A20 tumor was induced by means of the adoptive transfer of A20-specific Th1.

### Tumor Prevention with Tumor-reactive Th1

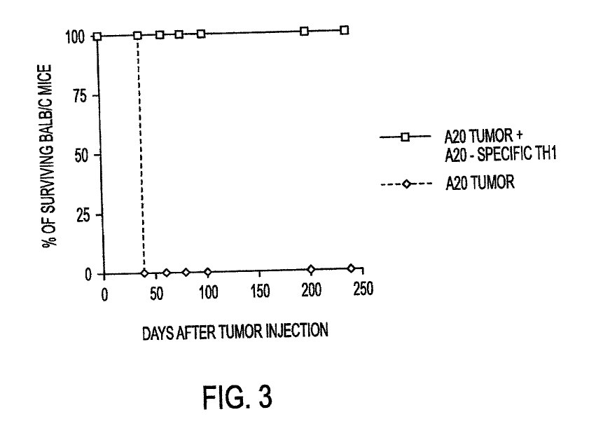

In tumor prevention experiments, 0.5×10⁶ A20 tumor cells and 0.5×10⁶ A20-specific Th1 were injected into Balb/c mice intraperitoneally, one directly after the other. By means of the adoptive transfer of the Th1 lines, the survival rates of the mice were significantly extended (FIG. 3). Depending on the experiment, between 70% and 100% of all the mice were completely healed. This was astounding in that the tumor is considered to be very aggressive and many traditional immune therapies that otherwise effectively allow tumor prevention fail when it comes to this tumor. A second important result was that an adoptive transfer of these Th1 lines protects against tumor development, but does not lead to autoimmune diseases: the animals can live under this protection until they die a natural death. Even after a year, the animals show no signs of tumors or autoimmune disease, either clinically or in an autopsy. Even if the animals become immune-suppressed, no tumors occur at a later point in time.

### Tumor Prevention with Tumor-reactive Th1

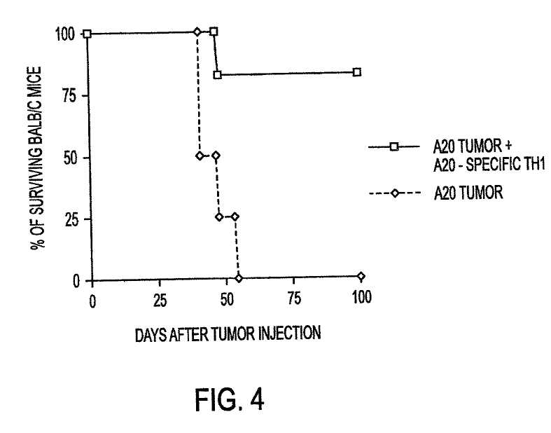

Based on these results, the Th1 were then used to treat tumors that were already established. 0.5×10⁶ A20 tumor cells were injected intravenously. On Day 7 after the transfer, a point in time at which large amounts of tumor can already be detected in the lymphoid organs, the animals again received an intravenous vaccination of 0.5×10⁶ A20-specific Th1 lines, in other words about 25×10⁶ Th1 cells per kg of body weight. The data from five experiments showed that (1) the tumor growth was significantly delayed in almost all the animals, and (2) up to 70% of the animals were considered healed by the transfer of the Th1 (FIG. 4). The therapy was still effective at a point in time at which immune therapies have not been described until now, and at which the tumor load of the spleen was about 100 times greater than for the immunization protocols that have been described until now.

Therefore the data in FIGS. 2, 3, and 4 show that adoptively transferred Th can provoke a very effective immune response to tumors. For the first time, it was shown, according to the invention, that

1) Th1 can be used very efficiently and safely, not only in prevention, but also in tumor therapy.

2) The therapy works even in immune-competent animals.

3) It can be assumed that Th1 can also be induced against cells of other tumors, particularly solid tumors, using the method described here, since the presentation of the antigens takes place predominantly via autologous/syngeneic APC (in this approach, spleen cells). The method thereby allows generation of effective Th1 also for an immune response to solid tumors that do not express MHC-II.

The attached figures explain the invention in greater detail.

FIG. 1 shows the production of IFN-γ and IL-4 by A20-specific Th1 cell lines after in vitro stimulation. The A20-specific IFN-γ production of a CD4+T cell line was generated in vitro with CpG-ODN and anti-IL-4 antibodies against the lymphoma, within two weeks. In this connection, CD4+T cells (Th) were isolated from Balb/c mice that had been immunized by an A20 tumor. The Th obtained were stimulated in the presence of syngeneic antigen-presenting cells (APC), A20 tumor cells, anti-IL-4-antibodies, IL-2 and CpG ODN 1668, and expanded over 10 days. On Day 11, the cultivated Th1 were restimulated for 24 hours in the presence of APC alone or additional A20 cells. The content of IL-4 and IFN-γ was determined from the top fraction of the culture.

FIG. 2 shows the lengthening of an A20-specific immune response by means of adoptive transfer of A20-specific Th1. The tumor-specific immune response to an A20-B-cell lymphoma is significantly extended by the adoptive transfer of A20-specific Th1. In this connection, Balb/c mice were immunized by means of intravenous injection of 0.5 million A20 tumor cells, alone or together with 0.5 million A20-specific Th1. On d5, 7, 10 the spleens of the mice were prepared and 1 million cells were incubated in vitro, with or without irradiated A20, for 48 hours, at 37° C. The IFN-γ was then determined from the top fraction of the culture.

FIG. 3 shows the prevention of an A20-B-cell lymphoma by means of adoptive transfer of A20-specific Th1. After adoptive transfer of the A20 lymphoma (tumor), the BALB/c mice die rapidly, and after administration of the A20 lymphoma and simultaneous, adoptive transfer of A20-specific Th1, long-term survival of the BALB/c mice occurs. In this connection, Balb/c mice were intravenously injected with 0.5 million A20 tumor cells alone, or simultaneously with 0.5 million A20-specific Th1, and the progression of therapy was checked visually.

FIG. 4 shows the treatment of an established A20-B-cell lymphoma by means of adoptive transfer of A20-specific Th1. After adoptive transfer of the A20 lymphoma, the BALB/c mice die rapidly, and up to ≧80% of the BALB/c mice recover in the long term if A20-specific Th1 were adoptively transferred seven days after administration of the A20 lymphoma. At this point in time, the tumor load is 100 times higher than for the most successful therapy of transferred tumor cells with cytokin. In this connection, Balb/c mice were intravenously injected with 0.5 million A20 tumor cells in 500 μl PBS. Seven days after the A20 injection (100 CFU/spleen), the mice were injected with 500 μl PBS alone or 0.5 million A20-specific Th1 in 500 μl PBS, and the progression of therapy was checked visually.

1. Process for the production of a pharmaceutical preparation, comprising tumor-specific Th cells from tumor cells with a cytokin pattern similar to Th1 , which demonstrate a high production of interferon-γ and produce little or no interleukin-4, where the ratio of the interferon-γ production to the interleukin-4 production is at least 10:1, together with pharmaceutically compatible excipients and processing aids, where tumor cells that have been inhibited in their proliferation by radiation or chemotherapeutics such as Mitomycin are cultivated in vitro together with autologous/syngeneic APC (antigen-presenting cells) and Th (CD4+T helper cells).

2. Process according to claim 1, characterized in that the production of interferon-γ amounts to at least 100 U/ml.

3. Process according to claim 1, characterized in that the production of interleukin-4 amounts to at least 1000 U/ml.

4. Process according to claim 1, characterized in that the ratio of the interferon-γ production to the interleukin-4 production amounts to at least 10:1.

5. Process according to claim 1, characterized in that the pharmaceutical preparation possesses a high specificity for tumor antigens.

6. Process according to claim 1, characterized in that the pharmaceutical preparation is present in a form suitable for adoptive transfer.
